(19) United States
(12) Patent Application Publication
(10) Pub. No.: US 2024/0298618 A1
(43) Pub. Date: Sep. 12, 2024

(54) UT2 GENE-DEFICIENT MOUSE MODEL AND METHOD FOR SCREENING THERAPEUTIC AGENT FOR MYELOID LEUKEMIA, USING SAME

(71) Applicant: PUSAN NATIONAL UNIVERSITY INDUSTRY-UNIVERSITY COOPERATION FOUNDATION, Busan (KR)

(72) Inventors: Dong Jun LEE, Yangsan-si (KR); Soo Yeon WOO, Yangsan-si (KR); Hee Seon CHOI, Yangsan-si (KR)

(73) Assignee: PUSAN NATIONAL UNIVERSITY INDUSTRY-UNIVERSITY COOPERATION FOUNDATION, Busan (KR)

(21) Appl. No.: 18/654,005

(22) Filed: May 3, 2024

Related U.S. Application Data
(63) Continuation of application No. PCT/KR2022/016874, filed on Nov. 1, 2022.

(30) Foreign Application Priority Data
Nov. 12, 2021 (KR) 10-2021-0155784
Oct. 27, 2022 (KR) 10-2022-0140290

(51) Int. Cl. A01K 67/0276; C12N 9/12; G01N 33/50

(52) U.S. Cl. CPC A01K 67/0276; C12N 9/1241; G01N 33/5017; A01K 2217/075; A01K 2227/105; A01K 2267/0331; G01N 2500/10

(57) ABSTRACT

The present disclosure relates to an upstream of mTORC2 (UT2) gene-deficient mouse model and a method of screening therapeutic agents for myeloid leukemia using the same, wherein it was found that the onset of myeloid leukemia and proliferation of myeloid leukemia cells were more facilitated in UT2 gene-deficient mice and UT2 genetic scissor (Cripsr/Cas9)-deficient myeloid leukemia cell lines. In addition, it was found that the survival rate of patients with low expression of UT2 was low in myeloid leukemia patients, while expression of UT2 was low in cells of actual myeloid leukemia patients.

### CROSS-REFERENCE TO RELATED APPLICATION

This application is a Continuation of PCT International Patent Application No. PCT/KR2022/016874 filed on Nov. 1, 2022, which claims priority to Korean Patent Application Nos. 10-2021-0155784 filed on Nov. 12, 2021 and 10-2022-0140290 filed on Oct. 27, 2022 which are all hereby incorporated by reference in their entirety.

### BACKGROUND OF THE INVENTION

The present application is supported by the National Research Foundation of Korea under Ministry of Science and ICT, Ministry of Trade, Industry and Energy, Ministry of Health & Welfare, Ministry of Food and Drug Safety for the following research projects:
1) A study on development of new concept erythrocyte differentiation and proliferation restoration technology through identification of differentiation dynamics of highly efficient mobilized hematopoietic stem progenitor cells based on niche enhancing factors (HX23C1692).
2) A study on research center for molecular control of cancer cell diversity (2022R1A5A2027161).
3) A study on mechanical study on cell plasticity and vascular regeneration of UT2, a novel cell surface inhibitor of mTORC2/AKT-STAT3 phosphorylation, in Leptin receptor-expressing stem cell niches (2021R1A2C4001466).
4) A study on Investigation on the role of UT2, novel cell surface inhibitor, for mTORC2-STAT3 signaling pathway in cardiovascular/hematopoietic stem cells (2018R1C1B6001290).

### 1. Field of the Invention

The present disclosure relates to an upstream of mTORC2 (UT2) gene-deficient mouse model and a method of screening therapeutic agents for myeloid leukemia using the same.

### 2. Description of the Related Art

It has been revealed that an upstream of mTORC2 (UT2) gene, a cell surface marker and a gene that regulates division and differentiation in hematopoietic stem cells, affects the mammalian mTORC2/AKT network and cytokine receptor/STAT3 signaling system.

Leukemia is a general term for diseases with neoplastic proliferation of white blood cells, types of which are divided into myeloid leukemia and lymphocytic leukemia depending on the white blood cells from which the leukemia originates, and also into acute leukemia and chronic leukemia depending on the progression rate. The clinical features of leukemia vary depending on the type of disease and the nature of the cells infiltrated. Lymphocytic leukemia is caused by mutations in lymphatic blood cells, myeloid leukemia by myeloid blood cells, and chronic myeloid leukemia by cells in the maturity stage, while acute myeloid leukemia is caused by disorders of myeloid blast cells that initiate differentiation in the relatively early stages of hematopoietic processes.

Hematopoietic stem cells maintain homeostasis by constantly producing new cells through division and differentiation, and patients with myeloid leukemia have a large number of abnormalities in hematopoietic stem cells and bone marrow, but the specific mechanism is still not clearly understood. In addition, although there are some reports on major genes capable of regulating the proliferative ability of hematopoietic stem cells and myeloid leukemia, the actual substantive issue is that there are limitations in models using gene-deficient mice and genetic scissors (Cripsr/Cas9).

### SUMMARY OF THE INVENTION

### Problem to be Solved by the Invention

An object of the present disclosure is to provide a UT2 gene-deficient transgenic mouse with a UT2 gene knocked out specifically for hematopoietic cells, prepared by crossing UT2 floxed mice and Mx1-Cre mice expressing Cre-recombinase specifically for hematopoietic cells, and a preparation method thereof.

Another object of the present disclosure is to provide a method of screening therapeutic agents for myeloid leukemia or proliferation stimulants for hematopoietic stem cells using the mouse model.

### Means for solving the Problem

To achieve the above objects, the present disclosure provides a UT2 gene-deficient transgenic mouse with a UT2 gene knocked out specifically for hematopoietic cells, prepared by crossing UT2 floxed mice and Mx1-Cre mice expressing Cre-recombinase specifically for hematopoietic cells.

Further, the present disclosure provides a method of preparing a hematopoietic cell-specific UT2 gene-deficient transgenic mouse, including: 1) crossing UT2 floxed mice and Mx1-Cre mice expressing Cre-recombinase specifically for hematopoietic cells; and 2) selecting mice with a UT2 gene knocked out specifically for hematopoietic cells from second-generation mice resulting from the crossing.

In addition, the present disclosure provides a method of screening therapeutic agents for myeloid leukemia, including: 1) treating the transgenic mouse according to the above with test materials; 2) measuring an indicator for a myeloid leukemia disease of the transgenic mouse treated with the test materials; and 3) selecting a test material with the improved indicator measured for the myeloid leukemia disease, compared with a control sample.

In addition, the present disclosure provides a method of screening proliferation stimulants for hematopoietic stem cells, including: 1) treating the transgenic mouse according to the above with test materials; 2) measuring a hematopoietic stem cell proliferative ability of the transgenic mouse treated with the test materials; and 3) selecting a test material with the improved hematopoietic stem cell proliferative ability measured, compared with a control sample.

### Effects of the Invention

The present disclosure relates to an upstream of mTORC2 (UT2) gene-deficient mouse model and a method of screening therapeutic agents for myeloid leukemia using the same, wherein it was found that onset of myeloid leukemia and proliferation of myeloid leukemia cells were more facilitated in UT2 gene-deficient mice and UT2 genetic scissor (Cripsr/Cas9)-deficient myeloid leukemia cell lines (HL60, THP1, KG1α, K562). In addition, it was identified that the survival rate of patients with low expression of UT2 was low in myeloid leukemia patients, while expression of UT2 was low in cells of actual myeloid leukemia patients. An increase in leukemia cells was identified as a decrease was shown in differentiation of UT2 genetic scissor (Cripsr/Cas9)-deficient myeloid leukemia cells into myeloid cells. It was found that, when UT2 gene-deficient mice were observed for hematopoietic stem cells (Mx1 Cre), the proliferative ability of hematopoietic stem cells was increased with a decrease shown in the resting phase, and a survival rate of UT2 gene-deficient mice decreased by increasing the proliferative ability of hematopoietic stem cells upon treatment of 5-FU anticancer drugs. The UT2 gene-deficient model according to the present disclosure may be useful in the study of pathology of myeloid leukemia, thereby enabling effective selection of therapeutic agents for myeloid leukemia using the same.

### BRIEF DESCRIPTION OF THE DRAWINGS

FIG. 1 shows preparation of a UT2 knock-out model and results of analyzing genotyping and expression levels thereof. A: Schematic design of target mutation for conditional knock-out of UT2, B: Genotyping of Ut2Fl/Fl, Mx1Cre; Ut2Fl/Fl mice, C: Expression of UT2 of bone marrow cells of Ut2FI/Fl, Mx1Cre; Ut2Fl/Fl mice, D: Analysis on frequency of T-ALL cells in T-ALL mice (PB: peripheral blood, BM: bone marrow, Sp: spleen), E: Analysis on pAKT^{S473} expression in T-ALL mice, F: Identification of UT2 knock-out in human T-ALL cell line Jurkat, G: Comparison of cell growth of UT2 knock-out Jurkat cells, H: Analysis on pAKT^{S473} expression of UT2 knock-out Jurkat cells, I: Tumor growth after xenotransplantation of UT2 knock-out Jurkat cells.

FIG. 2 shows results of analyzing effects of UT2 on myeloid leukemia. A: Survival curve according to UT2 expression in patients with acute myeloid leukemia (AML), B:

Identification of UT2 expression in normal individuals, AML patients, AML relapsed patients, and diffuse large B cell lymphoma (DLBCL) patients, C: Single cell RNA sequencing using samples of normal and AML patients (classified into 6 types including GMP, Prog, ProMono, HSC, cDC, and Mono), D: Frequency of cell types classified via single cell RNA sequencing, E: Analysis on expression levels of UT2 in monocytes and HSCs, F: Gene ontology with analyzed UT2-related genes in HSCs and cDCs. FIG. 3 shows results of analyzing effects of UT2 on AML by regulating expression of pAKT^{S473}. A: Identification of UT2 knock-out in human myeloid leukemia cell lines, B: Comparison of cell growth of UT2 knock-out myeloid leukemia cells, C, D: Analysis on PAKT^{S473} expression in UT2 knock-out myeloid leukemia cells, E: Analysis on a degree of differentiation of UT2 knock-out myeloid leukemia cells, F, G, H: Comparison of a degree of differentiation according to UT2 after xenotransplantation of HL60 (F), pAKT^{S473} expression (G), and viability (H), I: Comparison of a degree of differentiation and pAKT^{S473} expression depending on UT2 after transplantation of bone marrow cells infected with MLL-AF9 which is a human acute myeloid leukemia gene.

FIGS. 4 and 5 show results of analyzing a role of UT2 using UT2 knock-out mice. A of FIG. 4: Frequency of LKS and HPC in mouse bone marrow cells (LKS=Lin⁻Scal⁻cKit⁺ and HPC=Lin⁻Scal⁺cKit⁺CD48⁺), B of FIG. 4: Frequency of HSC in mouse bone marrow cells (HSC=Lin⁻Scal⁺cKit⁺CD150⁺CD48⁻), C of FIG. 4: Frequency of progenitor cells in mouse bone marrow cells; MEP=Lin⁻Scal⁻cKit⁺CD34⁻CD16/32; CMP=Lin⁻Scal⁻ cKit⁺CD34⁺CD16/32⁻; GMP=Lin⁻Scal⁻cKit⁺CD34⁺CD16/32; CLP=Lin⁻ Scal^{low}cKit^{low} CD127⁺. D of FIG. 4: Frequency of GO and S/G2/M phases in LKS, HPC, and HSC among mouse bone marrow cells, E of FIG. 4: Expression of pAKT^{S473} in LKS, HPC, and HSC among mouse bone marrow cells, F of FIG. 4: Identification of survival after injection of 5FU (150 mg/kg), G of FIG. 4: Identification of engraftment ability by bone marrow cell UT2 after bone marrow cell transplantation, H of FIG. 4: Identification of engraftment ability by bone marrow cell UT2 after bone marrow cell transplantation, I and J of FIG. 4: Determination of a ratio of donor bone marrow cells in LKS, HPC, HSC, and progenitor cells after bone marrow cell transplantation, K of FIG. 4: Expression levels of UT2 mRNA in donor bone marrow cells among bone marrow cells of recipient mice after bone marrow cell transplantation, L of FIG. 4: Identification of engraftment ability by Ut2 after second transplantation, M of FIG. 4: Identification of frequency of donor B cells, T cells, monocytes, and neutrophils in peripheral blood after second transplantation; B cells=B220⁺, T cell=CD3⁺, Monocyte=Mac1⁺Gr1⁻, Neutrophil=Mac1⁺Gr1⁺, N and O of FIG. 4: Determination of a ratio of donor bone marrow cells in LKS, HPC, HSC, and progenitor cells after second transplantation, P of FIG. 4: Identification of survival by UT2 in a graft-versus-host disease. A of FIG. 5: A count of mouse bone marrow cells, B of FIG. 5: Frequency of lineage cells in mouse bone marrow cells, C of FIG. 5: Weight of the mouse spleen, D of FIG. 5: Frequency of LKS, HPC, and HSC in mouse spleen cells, E of FIG. 5: Frequency of progenitor cells in mouse spleen cells, F of FIG. 5: A count of mouse spleen cells, G of FIG. 5: Frequency of lineage cells in mouse spleen cells, H of FIG. 5: Frequency of lineage cells in mouse peripheral blood, I of FIG. 5: Frequency of apoptosis of LKS, HPC, and HSC among mouse bone marrow cells, J of FIG. 5: Identification of engraftment ability by bone marrow cell Ut2 after bone marrow cell transplantation, K-M of FIG. 5: Determination of a ratio of donor bone marrow cells in lineage cells in the bone marrow, peripheral blood, and spleen after bone marrow cell transplantation, N of FIG. 5: Identification of engraftment ability by Ut2 after second transplantation, O of FIG. 5: Determination of a ratio of donor bone marrow cells in lineage cells after second transplantation.

FIG. 6 shows results of analyzing a role of UT2 in endothelial cells. A: Genotyping of Ut2Fl/Fl, Tie2Cre; Ut2Fl/Fl mice. B: Frequency of LKS and HPC in mouse bone marrow cells, C: Frequency of progenitor cells in mouse bone marrow cells, D: Frequency of lineage cells in mouse bone marrow cells, E: Identification of engraftment in the peripheral blood and lineage cells of peripheral blood after bone marrow cell transplantation, F: Identification of engraftment in the bone marrow and LKS, HPC, and HSC of bone marrow cells after bone marrow cell transplantation, G: Identification of engraftment of peripheral blood and lineage cells of peripheral blood after myeloid cell retrotransplantation, H: Identification of engraftment in LKS, HPC, and HSC in the bone marrow and bone marrow cells after myeloblast retrotransplantation.

FIG. 7 shows results of analyzing a role of UT2 in perivascular stromal cells. A: Genotyping of Ut2Fl/Fl, LepRCre; Ut2Fl/Fl mice, B: Frequency of LKS and HPC in mouse bone marrow cells, C: Frequency of progenitor cells in mouse bone marrow cells, D: Frequency of lineage cells in mouse bone marrow cells, E: Identification of engraftment in peripheral blood and lineage cells of peripheral blood after bone marrow cell transplantation, F: Identification of engraftment in LKS, HPC, and HSC of bone marrow and bone marrow cells after bone marrow cell transplantation, G: Identification of engraftment in peripheral blood and lineage cells of peripheral blood after myeloid cell retrotransplantation, H: Identification of engraftment in LKS, HPC, and HSC of bone marrow and bone marrow cells after myeloid cell retrotransplantation.

FIG. 8 shows results of analyzing a role of UT2 in osteoblasts. A: Genotyping of Ut2Fl/Fl, OncCre; Ut2Fl/Fl mice, B: Frequency of LKS and HPC in mouse bone marrow cells, C: Frequency of progenitor cells in mouse bone marrow cells, D: Frequency of lineage cells in mouse bone marrow cells, E: Identification of engraftment in peripheral blood and lineage cells of peripheral blood after bone marrow cell transplantation, F: Identification of engraftment in LKS, HPC, and HSC of the bone marrow and bone marrow cells after bone marrow cell transplantation, G: Identification of engraftment in peripheral blood and lineage cells of peripheral blood after myeloid cell retrotransplantation, H: Identification of engraftment in LKS, HPC, and HSC of the bone marrow and bone marrow cells after myeloid cell retrotransplantation.

### DETAILED DESCRIPTION OF THE INVENTION

The present disclosure provides a UT2 gene-deficient transgenic mouse with a UT2 gene knocked out specifically for hematopoietic cells, prepared by crossing UT2 floxed mice and Mx1-Cre mice expressing Cre-recombinase specifically for hematopoietic cells.

Preferably, the transgenic mouse may be a myeloid leukemia disease model, but is not limited to.

As used herein, “upstream of mTORC2 (UT2)” may have a gene ID 145407.

In addition, the present disclosure provides a method of preparing a hematopoietic cell-specific UT2 gene-deficient transgenic mouse, including: 1) crossing UT2 floxed mice and Mx1-Cre mice expressing Cre-recombinase specifically for hematopoietic cells; and 2) selecting mice with a UT2 gene knocked out specifically for hematopoietic cells from second-generation mice resulting from the crossing.

As used herein, the term “Cre/Lox genetic system” refers to a powerful system that regulates expression of two types of gene (Cre and Lox) derived from the PI bacteriophage and is a well-established research tool, especially in the field of mouse transformation. Cre recombinase catalyzes site-specific recombination between two loxP sites, which may be located on the same or separate DNA fragments. The two 13 bp repeat sequences on a single loxP site recognize and bind with the Cre protein to form a dimer, and the two loxP sites align in parallel to form a tetramer with four Cre proteins. Double-stranded DNA is cleaved out within the core spacer of each loxP site, and the two strands are ligated and intersect to cause deletion in the DNA, resulting in a transgenic animal with the desired DNA.

In addition, the present disclosure provides a method of screening therapeutic agents for myeloid leukemia, including: 1) treating the transgenic mouse according to the above with test materials; 2) measuring an indicator for a myeloid leukemia disease of the transgenic mouse treated with the test materials; and 3) selecting a test material with the improved indicator measured for the myeloid leukemia disease, compared with a control sample.

Preferably, the indicator for the myeloid leukemia disease may be frequency of Lin+Scal+cKit+(LSK), hematopoietic progenitor cells (HPCs), hematopoietic stem cells (HSCs), megakaryocyte-erythroid progenitors (MEPs), common myeloid progenitors (CMPs), granulocyte-macrophage progenitors (GMPs), or common lymphoid progenitors (CLPs) in bone marrow cells, or frequency of GO and S/G2/M phases of LKS, HPC, and HSC in bone marrow cells, but are not limited to.

In addition, the present disclosure provides a method of screening proliferation stimulants for hematopoietic stem cells, including: 1) treating the transgenic mouse according to the above with test materials; 2) measuring a hematopoietic stem cell proliferative ability of the transgenic mouse treated with the test materials; and 3) selecting a test material with the improved hematopoietic stem cell proliferative ability measured, compared with a control sample.

The term “test material” as used herein in reference to the screening method of the present disclosure refers to an unknown candidate substance that is used in screening to test whether it affects an expression level of genes or affects the expression or activity of a protein. The sample includes, but is not limited to, chemicals, nucleic acids, antisense-RNA, small interference RNA (siRNA), and natural extracts.

### [EXAMPLES]

Hereinafter, the present disclosure will be described in detail through example embodiments to help understanding of the present disclosure. The following example embodiments are provided to more completely explain the present disclosure to those of ordinary skill in the art and are merely illustrative of the content of the present invention, so the scope of the present disclosure is not limited to the following example embodiments.

### <Example 1> Preparation and Designing of UT2 Knock-out Models

1. Experimental method

1) Genotyping of mice

Genomic DNA is extracted from the mouse tail, the primer of DNA desired to be amplified is inserted, and the DNA is amplified through polymerase chain reaction (PCR), followed by detection via electrophoresis.

2) Analysis of UT2 expression levels

Bone marrow is extracted from the mouse tibia and thigh bones, and red blood cells are lysed to isolate bone marrow cells. After isolating RNA from the bone marrow cells, cDNA is synthesized to amplify DNA using a UT2 primer.

3) Transplantation of bone marrow cells

Bone marrow is extracted from the tibia and thigh bones of UT2Fl/Fl mice and Mx1Cre UT2Fl/Fl mice. Then, the T-ALL cells prepared by infection with retrovirus that induces human T-ALL are transplanted into irradiated wild-type mice. Afterwards, cells were extracted from the peripheral blood, bone marrow, and spleen of the transplanted mice, followed by flow cytometry.

4) Indel

To check UT2 knock-out from human leukemia cell lines, genomic DNA is extracted and amplified using polymerase chain reactions. The DNA is then treated with T7 endonuclease enzymes to cleave mismatched sites, followed by detection via electrophoresis.

2. Experimental results

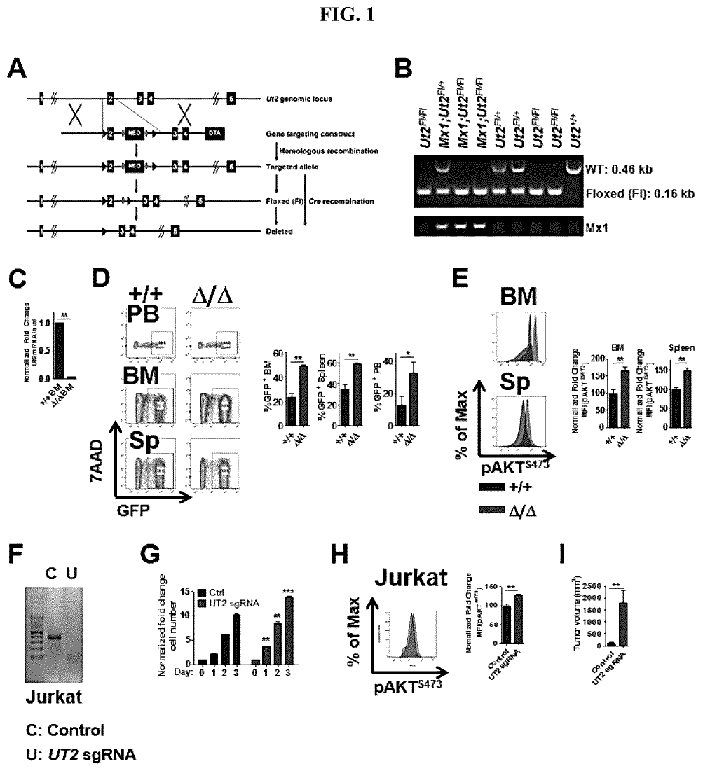

UT2 inhibits mTORC2 activity. mTORC2 regulates proliferation and survival of cells by regulating the activation of AKT, a key signaling substance responsible for PI3K signaling. These mTORC2s are highly involved in cancers, and PI3K/AKT-induced cancers rely on mTORC2 activity. Since UT2 reduces AKT activity by inhibiting mTORC2 activity, a conditional knock-out mouse model was prepared to proceed an experiment in order to determine whether UT2 affects the development of T-cell acute lymphoblastic leukemia (T-ALL) which is known to be sensitive to mTORC2. Conditional knock-out mice are typically used to study the function of specific genes in hematopoiesis, and Mx1-Cre knocks out the desired gene in hematopoietic cells as Cre is expressed by interferon. Mx1Cre; Ut2Fl/Fl mice (Δ/Δ) were prepared via crossing of Floxed UT2 mice with Mx1-Cre mice (FIG. 1A), and the mice's genes were identified through genotyping (FIG. 1B). UT2FI/Fl mice (+/+) were used as a control. Mx1Cre; Ut2Fl/Fl mice were administered with polyinosinic: polycytidylic acid (pipC) to delete UT2 by expressing cre recombinase, and as a result of detecting the level of UT2 mRNA in mouse bone marrow cells, it was found that UT2 was deleted (FIG. 1C). Next, bone marrow cells were extracted from the Ut2Fl/Fl mice and the Mx1Cre; Ut2Fl/Fl mice, infected with retrovirus that induces human T-ALL, respectively, and then transplanted to irradiated recipient mice, and as a result of analyzing T-ALL cells (GFP+cells) in the blood (PB), bone marrow (BM), and spleen (Sp) of recipient mice, it was found that T-ALL cells increased significantly in mice transplanted with UT2 knock-out T-ALL cells (FIG. 1D). In addition, the expression of pAKT^{S473} was also high in UT2 knock-out T-ALL cells (FIG. 1E).

In the Jurkat cell, a human T-ALL cell line, UT2 was knocked out using CRISPR small guide RNA. UT2 knock-out was identified by T7E1 test (FIG. 1F). As a result of UT2 knock-out, a growth rate of Jurkat cells increased (FIG. 1G), and the expression of pAKT^{S473} also increased (FIG. 1H). In addition, as a result of subcutaneously injecting UT2 knock-out Jurkat cells into athymic mice to generate tumors and measure their size, UT2 knock-out promoted tumor growth (FIG. 1I). These results indicate that UT2 affects T-ALL by regulating expression of pAKT^{S473}.

### <Example 2>Analysis on Effects of UT2 on Myeloid Leukemia

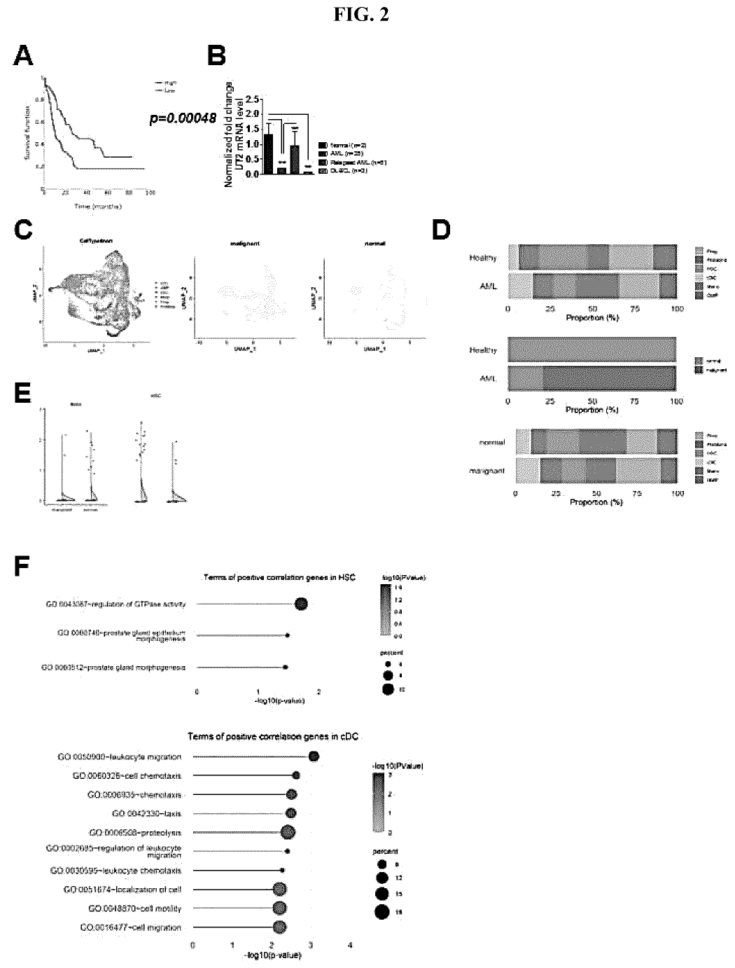

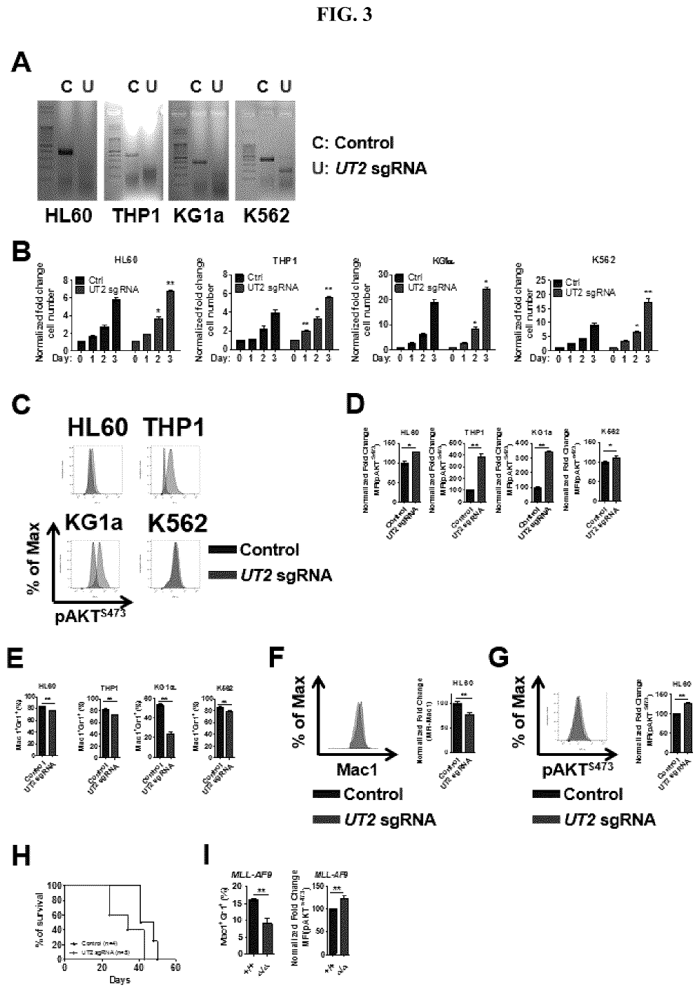

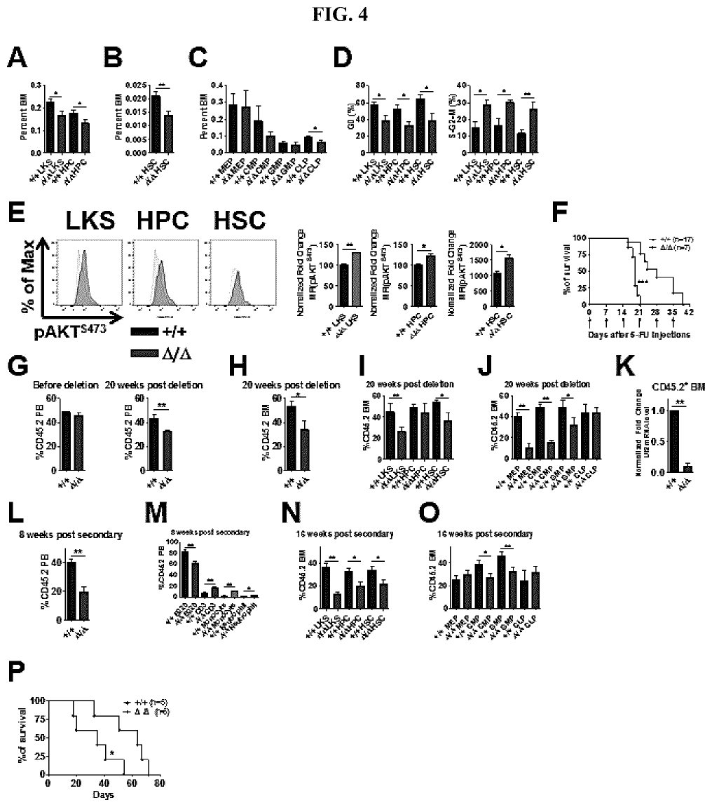

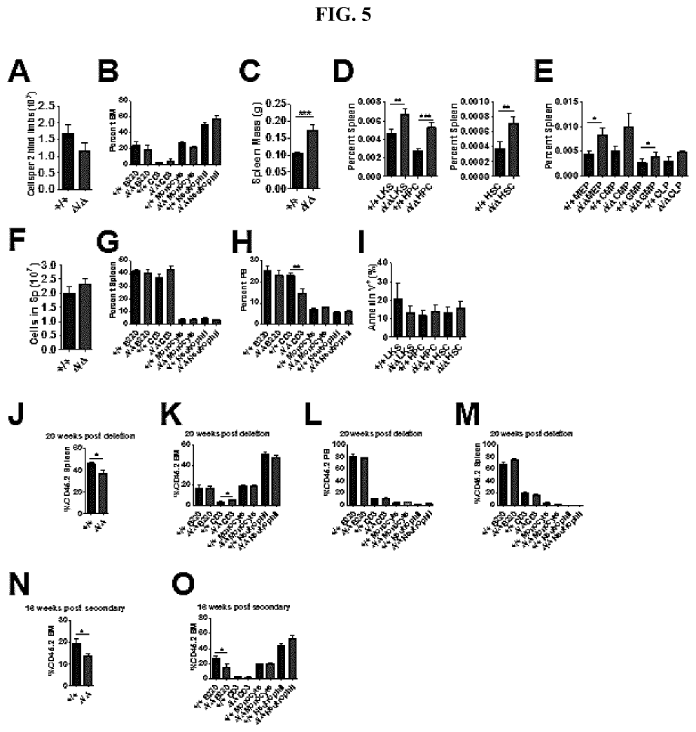

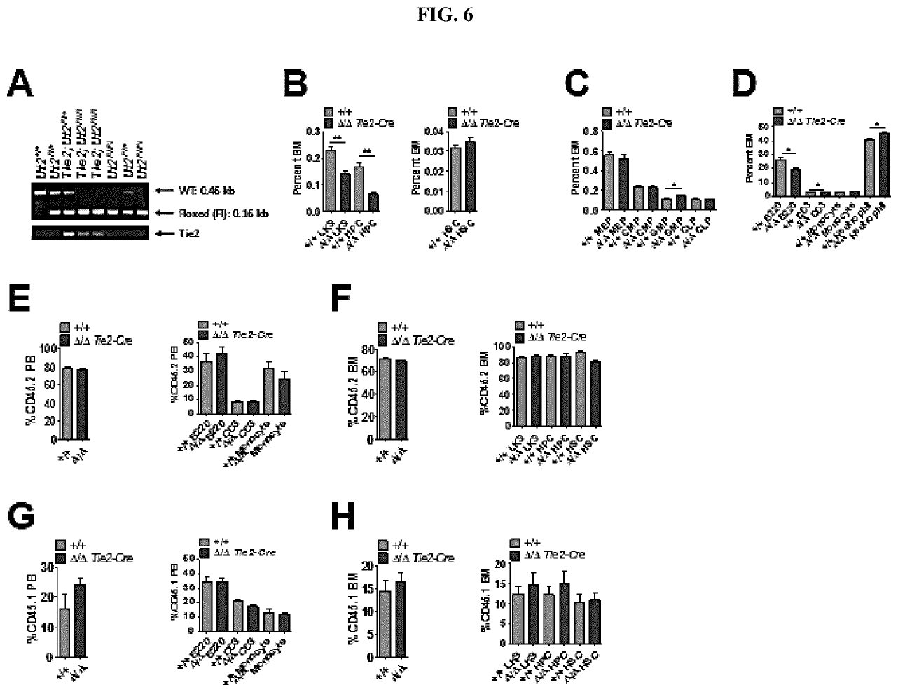

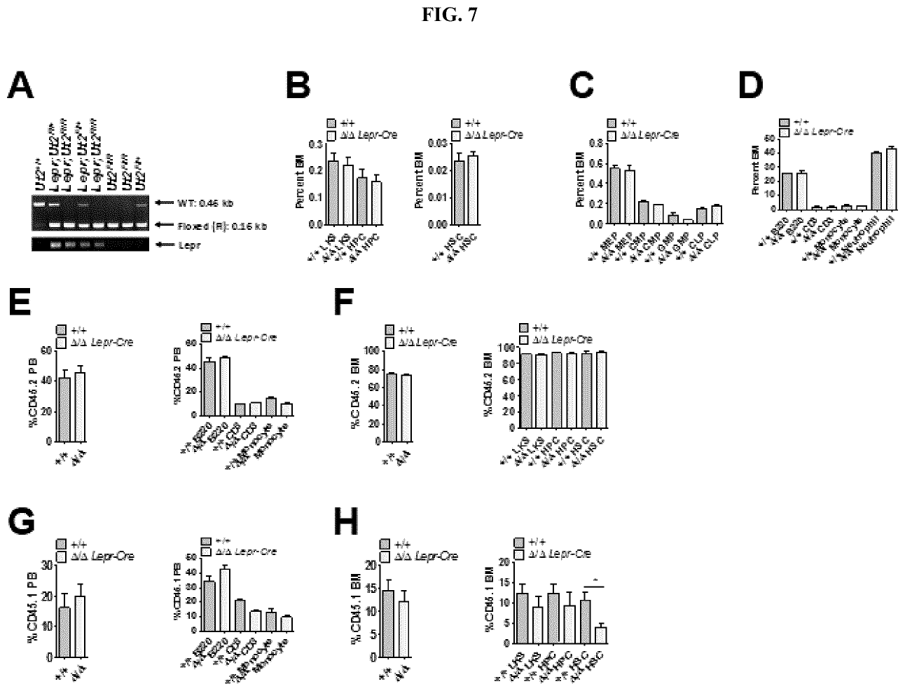

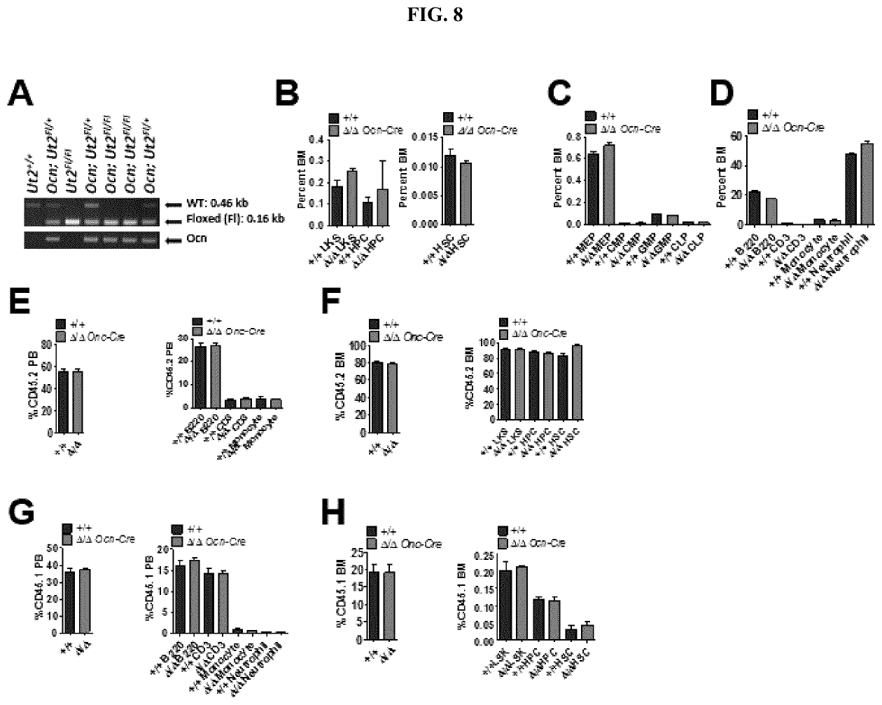

As AKT is important in proliferation and differentiation associated with myeloid malignancies, UT2 was expected to have an impact on myeloid leukemia. As a result of checking the overall survival rate in patients with acute myeloid leukemia (AML) according to UT2 expression levels, it was found that the survival rate increased with higher UT2 expression (FIG. 2A), and as a result of identifying the UT2 expression in patients' bone marrow cells, UT2 was reduced in bone marrow cells of AML patients and diffuse large B-cell lymphoma

(DLBCL) patients compared to bone marrow cells of normal individuals (FIG. 2B). On the other hand, UT2 increased in the bone marrow of patients with relapsed AML.

Single cell RNA sequencing was performed to observe UT2 expression at a single-cell level in AML patients (FIG. 2C). As a result of determining cell types based on gene expression for hematopoietic stem and progenitor cells (HSPCs) and lineage cells, compared to healthy donors, monocytes (mono) and dendritic cells (cDCs) in AML patients were present in higher proportions with the lower proportion of hematopoietic stem cells (HSCs) (Top of FIG. 2D). In addition, the proportion of malignant cells was higher in AML patients than in healthy donors (Middle of FIG. 2D), and in the case of malignant cells compared to normal cells, cDC to progenitor cells (Prog) were present in the higher proportion (Bottom of FIG. 2D). As a result of analyzing UT2 expression levels in AML patients, UT2 showed low expression in monocytes of AML patients (FIG. 2E). Gene ontology analysis is analysis on UT2-related genes in HSC and cDC, presenting association with genes related to immune response and metabolism (FIG. 2B). These results suggest that UT2 expression is reduced in patients with AML, indicating that UT2 is associated with the pathogenesis of hematologic malignancies.

### <Example 3>Evaluation of an Impact by UT2 on AML by Regulating Expression of pAKT^{S473}

1. Experimental Method 1) Flow cytometry (Identification of pAKT^{S473} expression)

After fixation of cells with 1% paraformaldehyde, cell membrane permeability is increased using 95% methanol. Flow cytometry is then performed by combining pAKT^{S473} antibodies and then secondary antibodies targeting pAKT^{S473}.

2) Flow cytometry (Identification of a degree of differentiation)

Flow cytometry is performed by adding cells into a FACS staining buffer and then combining Mac1, a monocyte antibody, and Gr1, a granulocyte antibody.

3) Xenotransplantation

UT2 knock-out HL60, which is prepared with sgRNA lentivirus to knock out UT2, is injected and transplanted into the vein of the tail of irradiated immunodeficient (NOD-SCID) mice. Afterwards, mouse peripheral blood is collected, red blood cells are lysed, and lymphocytes are collected to check expression and a degree of differentiation of pAKT^{S473}.

4) Transplantation of MLL-AF9 transformed bone marrow cells

Bone marrow is extracted from the tibia and thigh bones of UT2FI/Fl mice and Mx1Cre UT2Fl/Fl mice. AML cells, which are prepared by infection with the MLL-AF9 retrovirus that causes human AML, are then transplanted into irradiated wild-type mice.

2. Experimental Eesults

UT2 was knocked out using CRISPR small guide RNA in human myeloid leukemia cell lines HL60, THP1, KG1α, and K562. UT2 knock-out was identified by the T7E1 test (FIG. 3A). As a result of UT2 knock-out, the cell growth rate increased (FIG. 3B), and the expression of pAKT^{S473} also increased (FIGS. 3C-D). Differentiation of bone marrow cells was found to reduce leukemia cells and improve the survival rate of leukemia patients. As a result of determining the degree of differentiation in accordance with UT2 expression in myeloid leukemia cell lines by the Mac1+Gr1+cell ratio, Mac1+Gr1+cells decreased in UT2 knock-out myeloid leukemia cell lines (FIG. 3E). This suggests that the differentiation was reduced due to UT2 knock-out. HL60, a myeloid leukemia cell, was transplanted into a NOD-SCID mice which are immunodeficient mice. As a result, mice transplanted with UT2 knock-out HL60 reduced bone marrow differentiation (FIG. 3F), increased expression of pAKT^{S473} (FIG. 3G), and decreased survival rate (FIG. 3H). Next, to determine the anti-leukemia and differentiation effects of UT2, MLL-AF9, a human leukemia gene, was injected into bone marrow cells of Ut2Fl/Fl mice and Mx1Cre; Ut2Fl/Fl mice to be transplanted into irradiated recipient mice. As a result, with low proportion of Mac1+Gr1+cells in the bone marrow of mice transplanted with bone marrow cells from Mx1Cre; Ut2Fl/Fl mice, bone marrow cells failed to differentiate (Left of FIG. 31), and expression of pAKT^{S473} was also increased (Right of FIG. 31). These results indicate that UT2 affects AML by regulating the expression of PAKT^{S473}.

### <Example 4>Analysis on a Role of UT2 by UT2 Knock-out Mice

1. Experimental method

1) Transplantation of bone marrow cells

Bone marrow is extracted from the tibia and thigh bones of UT2FI/Fl mice and Mx1Cre UT2Fl/Fl mice. By mixing with bone marrow cells from CD45.1 mice in a 1:1 ratio, transplantation was performed in irradiated wild-type mice. After that, the bone marrow cells of the recipient mouse are extracted, followed by a second transplantation.

2) Graft-versus-host disease

Bone marrows are extracted from the tibia and thigh bones of UT2FI/Fl mice and Mx1Cre UT2Fl/Fl mice and then mixed with spleen cells from wild-type mice so as to be transplanted into irradiated Balb/C mice.

2. Experimental Results

As a result of analyzing bone marrow cells of Ut2Fl/Fl mice (+/+) and Mx1Cre; Ut2Fl/Fl mice (UT2 knock-out mice; Δ/Δ), Lin⁻Scal⁻cKit⁺(LSK), hematopoietic progenitor cells (HPCs), hematopoietic stem cells (HSCs), and common lymphoid progenitors (CLPs) were reduced in UT2 knock-out mice (FIGS. 4A-C), but there was no difference in cell counts and lineage cells (FIGS. 5A-B). In addition, the mass of the spleen (Sp) increased, LKS, HPC, and HSC increased as opposed to the bone marrow, and the megakaryocyte-erythroid progenitors (MEPs), common myeloid progenitors (CMPs), and granulocyte-macrophage progenitors (GMPs) increased (FIGS. 5C-E), with no difference in the cell counts and lineage cells (FIGS. 5F-G). In addition, there was no difference in lineage cells other than T cells (CD3) in peripheral blood (PB) (FIG. 5H). LSK, HPC, and HSC in UT2 knock-out mice were reduced dormant cells in the G0 stage, while cells in the S/G2/M stage in the dividing state were increased (FIG. 4D). In order to determine whether reduction in LKS, HPC, and HSC in UT2 knock-out mouse bone marrow is associated with apoptosis, apoptosis was observed using annexin V, but there was no difference (FIG. 51). Expression of pAKT^{S473} was higher in LSK, HPC, and HSC in UT2 knock-out mice (FIG. 4E). 5-fluorouracil (5-FU) is a type of anticancer drug that causes damage to DNA. When 5FU was injected into mice at 1-week intervals, the survival rate of UT2 knock-out mice was reduced (FIG. 4F).

To investigate the role of UT2 in HSC regeneration, bone marrow cells from Ut2FI/Fl mice expressing a CD45.2 gene and Mx1Cre; Ut2Fl/Fl mice were mixed with bone marrow of mice expressing the CD45.1 gene in a 1:1 ratio and transplanted into irradiated CD45.1 recipient mice. Recipient mice were injected with pipC for UT2 knock-out 4 weeks after transplantation, and as a result of analyzing peripheral blood for 20 weeks, it was found that engraftment of donor bone marrow cells was reduced in mice transplanted with bone marrow cells from Mx1Cre; Ut2Fl/Fl mice (UT2 knock-out mice) (FIG. 4G). In addition, bone marrow engraftment in UT2 knock-out mice was also reduced in the bone marrow and spleen (FIG. 4H, FIG. 5J), and the proportion of LKS, HPC, HSC, MEP, CMP, and GMP decreased (FIGS. 4I-J). However, the lineage cells did not differ all in bone marrow, peripheral blood, and spleen (FIGS. 5K-M). As a result of identifying expression of Ut2 mRNA by isolating CD45.2 bone marrow cells of donor mice from bone marrow cells of recipient mice, there was a decrease in mice transplanted with bone marrow cells from UT2 knock-out mice (FIG. 4K). Next, bone marrow cells from primary recipient mice were transplanted into secondary recipient mice, and it was found that the bone marrow regenerative ability of UT2 knock-out mice decreased in peripheral blood and bone marrow (FIGS. 4L-O, FIGS. 5N-O).

Hematopoietic stem cell transplantation is used as a treatment to treat leukemia. The transplanted immune cells are usually destructed by the recipient's immune mechanism, and in the case of reduced immune function, the graft-versus-host disease may occur, in which the transplanted immune cells attack the recipient. To investigate the role of UT2 in these graft-versus-host disease, bone marrow cells of Ut2Fl/Fl mice and Mx1Cre; Ut2Fl/Fl mice were mixed with spleen cells from wild-type mice and transplanted into mice of another species (Balb/c) so as to determine the survival rate by inducing the graft-versus-host disease. As a result, it was revealed that the survival rate of recipient mice transplanted with bone marrow cells from UT2 knock-out mice was reduced (FIG. 4P).

### <Example 5>Analysis on the role of UT2 in endothelial cells, perivascular stromal cells, and osteoblasts

1. Experimental method

1) Retrotransplantation of bone marrow cells

Bone marrow cells are extracted from the tibia and thigh bones of CD45.1 wild-type mice and transplanted into irradiated UT2Fl/Fl mice and Cre UT2FI/F1 (UT2 knock-out) mice.

2. Experimental results

Hematopoiesis, which is to make blood cells, takes place in the bone marrow. The bone marrow is made up of a variety of stromal cells, including hematopoietic stem cells, and is surrounded by bones with abundant blood vessels and high innervation. Hematopoietic stem cells perform hematopoietic action by interacting with various cells present in the microenvironment in the bone marrow. First, based on the study results in that endothelial cells surrounding blood vessels in the bone marrow play an important role in maintaining hematopoietic stem and progenitor cells, Tie2-Cre mice that act on endothelial cells were used to determine the role of UT2 in endothelial cells. Tie-Cre; Ut2Fl/Fl mice (A/A) were prepared via crossing of Floxed UT2 mice with Tie2-Cre mice, and the mouse genes were identified through genotyping (FIG. 6A). UT2FI/Fl mice (+/+) were used as a control. Of the bone marrow cells of Tie-Cre; Ut2Fl/Fl mice, LKS and HPC decreased (FIG. 6B), GMP increased (FIG. 6C), and changes were observed in B cells, T cells, and neutrophils (FIG. 6D). In order to investigate the role of UT2 in endothelial cells in HSC regeneration under stressful conditions, bone marrow cells from Ut2Fl/Fl mice expressing a CD45.1 gene and Tie2Cre; Ut2Fl/Fl mice were mixed with mouse bone marrow expressing the CD45.1 gene in a 1:1 ratio and transplanted into irradiated CD45.2 recipient mice. There was no difference in donor cell engraftment in the peripheral blood and bone marrow of transplanted mice (FIGS. 6E-F). In addition, to investigate whether UT2 affects engraftment of transplanted bone marrow cells in the microenvironment of bone marrow, as a result of subjecting Ut2Fl/Fl mice and Tie2Cre; Ut2Fl/Fl mice to irradiation to transplant bone marrow cells of CD45.1 mice, there was also no difference in engraftment of donor cells (FIGS. 6G-H).

Next, LeprCre mice acting on perivascular stromal cells were used to determine the role of UT2 in perivascular stromal cells. LeprCre; Ut2Fl/Fl mice (A/A) were prepared through crossing of Floxed UT2 mice and LeprCre mice, and the mice's genes were identified through genotyping (FIG. 7A). UT2Fl/Fl mice (+/+) were used as a control. There was no change in the bone marrow cells of the LeprCre; Ut2Fl/Fl mice (FIGS. 7B-D). To investigate the role of UT2 in perivascular stromal cells in HSC regeneration under stressful conditions, bone marrow cells from Ut2Fl/Fl mice expressing the CD45.2 gene and LeprCre; Ut2Fl/Fl mice were mixed with bone marrow of mice expressing CD45.1 gene in a 1:1 ratio and transplanted into irradiated CD45.2 recipient mice. There was no difference in donor cell engraftment in the peripheral blood and bone marrow of transplanted mice (FIGS. 7E-F). In addition, as a result of subjecting the Ut2Fl/Fl mice and LeprCre; Ut2Fl/Fl mice to irradiation to transplant bone marrow cells from CD45.1 mice, there was no difference in donor cell engraftment (FIGS. 7G-H).

Based on the study results in that osteoblasts are the cells responsible for osteogenesis and affect the regeneration of hematopoietic stem cells, OncCre mice that act on osteoblasts were used to determine the role of UT2 in the bone. OncCre; Ut2Fl/Fl mice (A/A) were prepared via crossing of Floxed UT2 mice with OncCre mice, and the mouse genes were identified through genotyping (FIG. 8A). UT2Fl/Fl mice (+/+) were used as a control. There was no change in the bone marrow cells of the OncCre; Ut2Fl/Fl mice (FIGS. 8B-D). To investigate the role of UT2 of osteoblasts in HSC regeneration under stressful conditions, bone marrow cells from Ut2Fl/F1 mice expressing the CD45.2 gene and OncCre; Ut2Fl/Fl mice were mixed with bone marrow from mice expressing the CD45.1 gene in a 1:1 ratio and transplanted into irradiated CD45.2 recipient mice. There was no difference in donor cell engraftment in the peripheral blood and bone marrow of transplanted mice (FIGS. 8E-F). In addition, as a result of subjecting the Ut2Fl/Fl mice and OncCre; Ut2Fl/Fl mice to irradiation to transplant bone marrow cells from CD45.1 mice, there was no difference also in donor cell engraftment (FIGS. 8G-H).

Having described in detail a specific part of the present disclosure above, it is clear to those of skilled in the art that this specific description is merely a preferred example embodiment, and the scope of the present disclosure is not limited thereby. In other words, the substantial scope of the present disclosure is defined by the appended claims and their equivalents.

1. A UT2 gene-deficient transgenic mouse with a UT2 gene knocked out specifically for hematopoietic cells, prepared by crossing UT2 floxed mice and Mx1-Cre mice expressing Cre-recombinase specifically for hematopoietic cells.

2. The transgenic mouse of claim 1, wherein the transgenic mouse is a myeloid leukemia disease model.

3. A method of preparing a hematopoietic cell-specific UT2 gene-deficient transgenic mouse, the method comprising:
1) crossing UT2 floxed mice and Mx1-Cre mice expressing Cre-recombinase specifically for hematopoietic cells; and
2) selecting mice with a UT2 gene knocked out specifically for hematopoietic cells from second-generation mice resulting from the crossing.

4. A method of screening therapeutic agents for myeloid leukemia, the method comprising:
1) treating the transgenic mouse according to claim 1 with test materials;
2) measuring an indicator for a myeloid leukemia disease of the transgenic mouse treated with the test materials; and
3) selecting a test material with the improved indicator measured for the myeloid leukemia disease, compared with a control sample.

5. The method of claim 4, wherein the indicator for the myeloid leukemia disease is frequency of Lin⁻Scal⁻cKit⁺(LSK), hematopoietic progenitor cells (HPCs), hematopoietic stem cells (HSCs), megakaryocyte-erythroid progenitors (MEPs), common myeloid progenitors (CMPs), granulocyte-macrophage progenitors (GMPs), or common lymphoid progenitors (CLPs) in bone marrow cells, or frequency of G0 and S/G2/M phases of LKS, HPC, and HSC in bone marrow cells.
